(19) United States
(12) Patent Application Publication
(10) Pub. No.: US 2014/0017203 A1
(43) Pub. Date: Jan. 16, 2014

(54) RECOMBINANT ADENOVIRUS EXPRESSING ALPHA-A-CRYSTALLIN GENE AND GENE THERAPY FOR RETINALVASCULAR DISEASE USING THE SAME

(71) Applicants: Wan Sung Choi, Gyeongsangnam-do (KR); Eun Mi Hwang, Gyeongsangnam-do (KR); Jae Yong Park, Gyeongsangnam-do (KR); Gyeong Jae Cho, Gyeongsangnam-do (KR); Young Hee Kim, Gyeongsangnam-do (KR); So Yun Park, Gyeongsangnam-do (KR)

(72) Inventors: Wan Sung Choi, Gyeongsangnam-do (KR); Eun Mi Hwang, Gyeongsangnam-do (KR); Jae Yong Park, Gyeongsangnam-do (KR); Gyeong Jae Cho, Gyeongsangnam-do (KR); Young Hee Kim, Gyeongsangnam-do (KR); So Yun Park, Gyeongsangnam-do (KR)

(73) Assignee: Industry-Academic Cooperation Foundation Gyeongsang National University, Gyeongsangnam-do (KR)

(21) Appl. No.: 13/980,876

(22) PCT Filed: Jan. 20, 2012
(86) PCT No.: PCT/KR2012/000580

§ 371 Date: Sep. 25, 2013

(30) Foreign Application Priority Data
Jan. 21, 2011 (KR) 1020110006447

(51) Int. Cl. A61K 35/76

(52) U.S. Cl. CPC A61K 35/761
USPC 424/93.2; 435/235.1

(57) ABSTRACT

The present invention relates to a recombinant adenovirus expressing an αA-crystallin gene, and gene therapy for retinal vascular disease using the recombinant adenovirus. Gene therapy using the recombinant adenovirus comprising an αA-crystallin gene of the present invention increases the expression level of the αA-crystallin gene in the damaged retinal pericytes to suppress pericyte loss and death, retinal vascular leakage, and leukocyte adhesion surrounding retinal vessels, thereby protecting the pericytes. Therefore, it can be used for the prevention and treatment of various retinal vascular diseases including diabetic retinopathy.

### BACKGROUND OF THE INVENTION

1. Field of the Invention

The present invention relates to a recombinant adenovirus expressing an αA-crystallin gene, and gene therapy for retinal vascular disease using the same. In particular, the present invention provides a recombinant adenovirus capable of effectively preventing or treating retinal vascular diseases by inducing αA-crystallin gene expression to suppress apoptosis of retinal pericytes, retinal vascular leakage, adhesion of leukocytes to the retinal blood vessels, and blood-retinal barrier breakdown in retinal vascular disease.

2. Description of the Related Art

In general, diabetes is a complex metabolic disease that causes microvascular complications. Diabetes is the cause of a wide range of systemic disorders, and in particular, is one of common systemic diseases affecting the eye. Of them, diabetic retinopathy (DR) is the major cause of blindness in diabetic patients, and early diabetic retinopathy is characterized by increased vascular permeability and. progressive vascular damage. Blood-retinal barrier (BRB) breakdown and consequent vessel leakage in the retina are the main events in the pathogenesis of diabetic retinopathy. Pericyte loss is considered a hallmark of early diabetic retinopathy, and plays an important role in vessel leakage and leukocyte adhesion. Therefore, prevention of pericyte loss is essential for maintaining normal retinal blood vessels, and pericytes are the primary therapeutic target for diabetic retinopathy. Pericyte loss is believed to damage the blood vessel, but the underlying mechanisms are currently unclear.

Diabetic retinopathy is largely divided into nonproliferative diabetic retinopathy and proliferative diabetic retinopathy. In nonproliferative diabetic retinopathy, the retinal blood vessels are blocked or the walls of retinal blood vessels are damaged, causing hemorrhages or leaking fluid, and leading to retinal ischemia and edema, thereby resulting in vision loss. As these symptoms further progress, unnecessary blood vessels grow in the retina, a process called “neovascularization”. This condition is called proliferative diabetic retinopathy. These new blood vessels cause severe bleeding inside the eye, and grow on the retina, along with fibrous tissue. Normally, the retina is under tension and lies flat against the inside wall of the eye. However, this neovascularization can cause the retina to wrinkle and be pulled from the inside wall, which is called tractional retinal detachment. The new blood vessels can also grow into the angle of the anterior chamber of the eye and block the normal flow of fluid out of the eye, causing neovascular glaucoma.

Patients diagnosed with diabetic retinopathy are at an increased risk for intraocular hemorrhage, and repeated treatments are usually needed. Since diabetic retinopathy often has no early warning signs, and macular edema and proliferative retinopathy may not have any signal symptoms, diabetic retinopathy can go unnoticed and progress into the more serious stage of the disease. A recent way to prevent diabetic retinopathy is only known to control the blood glucose, blood pressure, and cholesterol levels. Substantially, the prophylactic treatment will reduce the occurrence of visual impairment in diabetic patients, and reduce the progression of the disease. Accordingly, there is an urgent need to understand the cause and pathogenesis of retinal vascular diseases including diabetic retinopathy with accuracy, which makes it possible to develop a therapeutic agent for the prevention or treatment of these diseases and a treatment method thereof.

Meanwhile, α-crystallin is a chaperon gene belonging to the family of small heat-shock proteins (sHSPs) that perform many physiological functions, including the maintenance of cell survival. α-Crystallin, a predominant protein of the ocular lens, is composed of two subunits, αA and αB. αA-Crystallin is known to be expressed only in the lens and retina, while αB-crystallin is widely distributed in non-lenticular tissues. In general, hyperglycemia caused by diabetes induction up-regulates αA-crystallin, which affects cell protection against the hyperglycemia stress. This phenomenon also occurs in αB-crystallin. Most of these functions have been associated with disease progression, and substantially α-crystallins can be considered as targets to treat diseases. However, their physiological significance in the context of the pathological vasculature in diabetes remains unknown.

The present inventors have made many efforts to reveal the association between α-crystallin and diabetic retinopathy in an diabetes-induced animal model. As a result, they found that expression of αA-crystallin protein is significantly down-regulated along with the retinal vascular damage and breakdown caused by diabetic retinopathy, and symptoms associated with retinal vascular damage can be inhibited by compensating for such down-regulation using a recombinant adenovirus comprising an αA-crystallin gene, so as to prevent or treat retinal vascular diseases including diabetic retinopathy, thereby completing the present invention.

### SUMMARY OF THE INVENTION

An object of the present invention is to provide a recombinant adenovirus expressing an αA-crystallin gene.

Another object of the present invention is to provide a pharmaceutical composition for prevention or treatment of retinal vascular disease, the recombinant adenovirus expressing an αA-crystallin gene as an active ingredient.

Still another object of the present invention is to provide a method for preventing or treating the retinal vascular disease of a subject, comprising the step of administering the pharmaceutical composition comprising the recombinant adenovirus as an active ingredient to the subject.

### BRIEF DESCRIPTION OF THE DRAWINGS

FIG. 1 shows vascular leakage induced by diabetes. (A) shows visualization of retinal vessels in 2-month diabetic mice and control mice, in which the arrows indicate vessel leakage in diabetic retinas. (B) shows the results of Evans blue leakage assay, in which a 1.63-fold increase in Evans Blue leakage was observed in diabetic retinas, compared with controls (P=0.0046, n=10).

FIG. 2 shows the results of Western blotting for the effect of diabetes on PARP and caspase-3, in which expression levels of cleaved PARP and active caspase-3 were increased in the diabetic retinas, compared with controls (1.55-fold and 1.57-fold, respectively; P=0.001 and 0.045, n=6), in which the protein expression was normalized to α-tubulin content in the sample.

FIG. 3 shows retinal sections stained with α-SMA and TUNEL. As a result of double staining for the pericyte marker α-SMA and TUNEL, a stronger expression was observed, in diabetic retinas where two markers were merged (arrowheads).

FIGS. 4 to 6 show a reduced αA-crystallin expression in diabetes-induced pericytes, in which αA-crystallin expression was down-regulated in retinas of 2-month diabetic mice, compared with the control.

FIG. 4 shows 2-DE gel images and the result of MALDI-TOF analysis. (A) shows 2-DE gel images of the retinal protein after staining, in which total 30 μg of each retinal protein were used in 2-DE analysis, and two spots (arrows) were significantly decreased in diabetic retinas compared with controls. (B) shows the results of MALDI-TOF analysis, in which the reduced spots were identified, as αA-crystallin.

FIG. 5 shows the results of Western blotting for αA-crystallin, in which αA-crystallin was identified as two different isoforms, and both isoforms were remarkably reduced in diabetic retinas (40% reduction; P=0.0322, n=6). C represents a control group and D represents a diabetic group.

FIG. 6 shows retinal sections stained with αA-crystallin and α-SMA. As a result of double staining for αA-crystallin and the pericyte marker α-SMA, a stronger expression was observed in diabetic retinas when two markers were merged (arrowheads). NFL is an abbreviation for nerve fiber layer).

FIGS. 7 to 9 show that αA-crystallin-expressing adenovirus targets blood vessels, and suppresses αA-crystallin decrease in diabetic retinas.

FIG. 7 shows schematic diagrams of (A) recombinant adenovirus (rAd-GFP and rAd-αAC-GFP), and (B) the recombinant adenovirus injection in the animal model according to the present invention.

FIG. 8 snows fluorescent images of GFP expressed in the retinal blood vessels, in which the testing efficiency of the recombinant adenovirus at 0, 2, 4, 8 weeks after virus injection was confirmed via GFP fluorescence imaging in retinal vessels, and recombinant adenovirus-mediated GFP expression (arrowheads) was maintained over 10 weeks in retinal blood vessels after virus treatments.

FIG. 9 shows expressions of GFP and αA-crystallin after injection of recombinant adenovirus (rAd-GFP and rAd-αAC-GFP). The protein expression was confirmed by Western blotting (A), and the relative expression levels were quantified and compared (B). GFP was expressed in all retinas after injection of two types of virus. In the retinas at 2 weeks after induction of diabetes, decrease in αA-crystallin protein was strongly blocked by treatment of rAd-αAC-GFP, compared with rAd.-GFP (1. 798-fold; P=0.0046, n=5). Meanwhile, adenovirus did not affect αA-crystallin levels in control mice. C represents a control group and D represents a diabetic group.

FIGS. 10 to 12 show that the recombinant adenovirus expressing αA-crystallin protein protects the diabetes-induced increase in cell death proteins or the loss of retinal pericytes.

FIG. 10 shows the effects of adenovirus treatment on PARP and caspase-3 expressions. (A) shows the result of Western blotting of cleaved PARP and active caspase-3 in retinas of 2-month diabetic mice after treatment of recombinant adenovirus. (B) and (C) show the relative expression levels of cleaved PARP and active caspase-3, respectively. Cleaved PARP and active caspase-3 increased by diabetes were more effectively reduced by treatment of rAd-αAC-GFP, compared with rAd-GFP (4-fold and 3-fold, respectively; P=0.0027 and 0.0133, n=4).

FIG. 11 shows fluorescent images of retinas labeled with the pericyte marker. (A) shows images of diabetic retinal sections stained with α-SMA and TUNEL, in which the increased death of pericytes by diabetes was reduced by rAd-αAC-GFP treatment. (B) shows pericytes surrounding blood vessels by double staining of the whole retinas with TMR-D and α-SMA (pericyte marker) (arrowheads).

FIG. 12 shows the number of pericytes per unit blood vessel upon treatment of adenovirus. The number of pericytes per unit blood vessel (0.01 mm²) was lower in retinas of 2-month diabetic mice, than in controls (2.7-fold; P=0.024, n=5), but pericyte loss was more effectively blocked by treatment of rAd-αAC-GFP than rAd-GFP (2.02-fold; P=0.021, n=5).

FIG. 13 shows the results of Evans Blue leakage assay for examining the protective effect of αA-crystallin-expressing recombinant adenovirus on vessel leakage in diabetic retinas. As a result of Evans Blue leakage assay for BRB breakdown, retinas of 2-month diabetic mice showed higher BRB breakdown than controls (2.67-fold; P=0.018, n=4), but BRB breakdown increased in diabetic retinas was greatly reduced by treatment of rAd-αAC-GFP, compared with rAd-GFP (1.98-fold; P=0.046, n=6).

FIG. 14 shows protective effect of αA-crystallin-expressing recombinant adenovirus on leukocyte adhesion in diabetic retinas. (A) shows adherent leukocytes (arrowheads) photographed at ×800 magnification after in situ labeling with TRITC-coupled concanavalin A lectin. (B) shows retinal leukostasis. When the number of adherent leukocytes in the entire retinas (arrowheads of A) was counted, a 1.6-fold increase in leukocyte adhesion was observed in diabetic retinas, compared with the controls (P=0.0011, n=5), but rAd-αAC-GFP treatment did not affect leukocyte adhesion (P>0.05, n=5).

### DETAILED DESCRIPTION OF THE PREFERRED EMBODIMENTS

In one aspect to achieve the above object, the present invention provides a recombinant adenovirus expressing an αA-crystallin gene.

As used herein, the term “αA-crystallin” is a family of heat-shock proteins, and is a chaperon protein that performs many physiological functions, including the maintenance of cell survival.

In the present invention, the αA-crystallin protein refers to a αA-crystallin protein of SEQ ID NO. 3 (Genebank ID: AAH85172) or a protein having a physiological activity substantially equivalent to that of the αA-crystallin protein. The protein having the substantially equivalent physiological activity comprises the αA-crystallin protein of SEQ ID NO. 3 (Genebank ID: AAH85172), a functional equivalent thereof, and a functional derivative thereof.

The term “functional equivalent” includes amino acid sequence variants having substitutions in some or all of the amino acids of SEQ ID NO. 3 (Genebank ID: AAH85172), or deletions or additions in some of the amino acids, and refers to those having physiological activity substantially equivalent to that of the αA-crystallin protein of SEQ ID NO. 3 (Genebank ID: AAH85172).

The term “functional derivative” is a protein having a modification for increasing or decreasing physicochemical properties of the αA-crystallin protein, and refers to those having physiological activity substantially equivalent to that of the αA-crystallin protein of SEQ ID NO. 3 (Genebank ID: AAH85172).

In the present invention, the αA-crystallin gene is characterized in that it includes the nucleic acid sequence encoding the αA-crystallin protein or the functional equivalent thereof, and DNA, cDNA and RNA sequences are all included in the nucleic acid sequence. Preferably, the αA-crystallin gene is represented by the nucleic acid sequence encoding the amino acid sequence of SEQ ID NO. 3 (Genebank ID: AAH85172), and most preferably, represented by the nucleic acid sequence of SEQ ID NO. 4 (Genebank ID: BC085172).

As used herein, the term “adenovirus” refers to a non-enveloped, icosahedral DNA virus of 60 to 85 nm in diameter. In general, adenovirus is known as an excellent gene delivery vector for animal cells, because of its high efficiency of gene transfer, ability to transfer genes into undifferentiated cells, and easy preparation of high titer viral stocks, and clinical trials have been conducted using adenovirus as a vector for gene therapy.

As used herein, the term “recombinant adenovirus” refers to a recombinant adenovirus that is prepared by comprising a gene encoding the heat shock protein, αA-crystallin, preferably, a gene having the nucleic acid, sequence of SEQ ID NO. 4 (Genebank ID: BC085172) in the adenovirus, and then is introduced into a host cell to express αA-crystallin.

The adenovirus usable in the recombinant adenovirus of the present invention may include various types, for example, type 1, type 2, type 3, type 4, type 5 or the like, and most preferably type 5. The adenovirus used as the recombinant adenovirus of the present invention is preferably replication-incompetent. E1A gene is known to be essential for adenoviral replication. Therefore, the replication-incompetent, recombinant adenovirus used as the recombinant adenovirus of the present invention is E1A gene-deleted or has mutations in the E1A gene for defective replication. The recombinant adenovirus of the present invention is introduced into a target cell with high transduction efficiency, and then expresses an αA-crystallin gene therein, thereby preventing or treating retinal vascular diseases including diabetic retinopathy with improved efficacy.

The recombinant adenovirus of the present invention may comprise a gene encoding the αA-crystallin protein or the functional equivalent thereof, a promoter operably linked to the gene, and a polyadenylatron signal.

As used herein, the term “operably linked” refers to an arrangement of elements that allows the elements to perform their usual function. In other words, it means that the coding sequence is fused to a promoter, an enhancer, a terminator sequence or the like so that the coding sequence is faithfully transcribed, spliced/joined, and translated and other structural features allow them to perform their individual functions. Therefore, a predetermined gene expression regulatory unit, in particular, a promoter operably linked to a coding sequence (e.g., a sequence encoding a desired protein) directs expression of the coding sequence in the presence of appropriate enzyme. The promoter or other regulatory elements need not be contiguous with the coding sequence, as long as they function to direct the expression thereof. For example, intervening untranslated yet transcribed sequences can be present between the promoter sequence and the coding sequence, and the promoter sequence can still be considered “operably linked” to the coding sequence. The operable linkage to a recombinant vector may be prepared using a genetic recombinant technique well known in the art, and site-specific DNA cleavage and ligation may be achieved using restriction and ligation enzymes generally known in the art. The promoter usable in the recombinant adenovirus of the present invention may be CMV (Cytomegalovirus), but not limited thereto.

Also, the recombinant adenovirus of the present invention may further comprise a secretory signal sequence for extracellular secretion of the recombinant protein. The secretory signal sequence is preferably any sequence known in the art, and most preferably, a signal sequence of granulocyte-macrophage colony stimulating factor (‘GM-CSF’) or preprotrypsin enzyme.

In addition, the recombinant adenovirus of the present invention may further comprise a reporter gene.

As used herein, the “reporter gene” refers to a nucleic acid encoding an identifying factor that is able to be screened based on the reporter gene's effect, in which the effect is used to track the inheritance of a nucleic acid of interest, to identify a cell or organism that has inherited the nucleic acid of interest, and/or to measure gene expression induction or transcription. The reporter genes usable in the present invention can be any reporter gene known in the art, and for example, may include green fluorescent protein (GFP), modified green fluorescent protein (mGFP), enhanced green fluorescent protein (EGFP), red fluorescent protein (RFP), modified red fluorescent protein (mRFP), enhanced red fluorescent protein (ERFP), blue fluorescent protein (BFP), enhanced blue fluorescent protein (EBFP), yellow fluorescent protein (YFP), enhanced yellow fluorescent protein (EYFP), cyan fluorescent protein (CFP), enhanced cyan fluorescent protein (ECFP) or the like. Selectable marker genes may be also considered as the reporter gene.

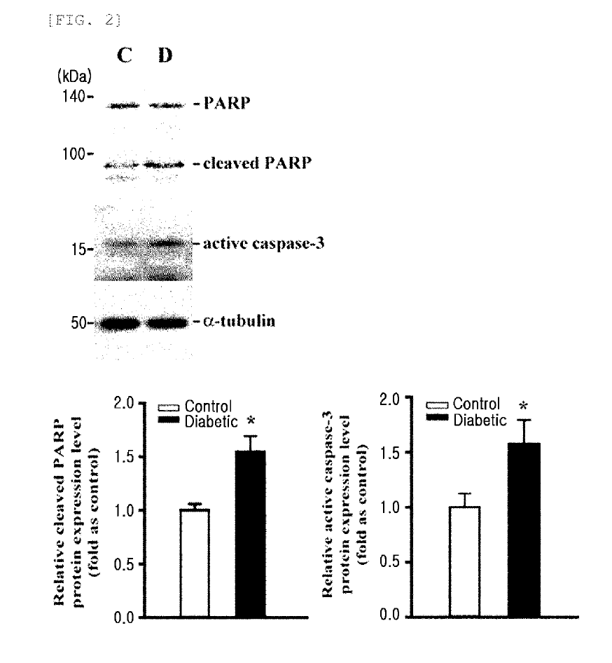

In the preferred Example of the present invention, a recombinant adenovirus rAD-αAC-GFP comprising a nucleic acid sequence encoding the mouse-derived, wild-type αA-crystallin protein, a CMV promoter, a GFP reporter gene, and a polyadenylation signal is prepared (see FIG. 2A). This recombinant adenovirus was deposited at the gene bank (KCTC) of Korea Research Institute of Bioscience and Biotechnology (KRIBB) on Jan. 6, 2011, and endowed with the deposition number of KCTC 11844BP.

Because the recombinant adenovirus of the present invention is deficient in the E1 gene that is essential for viral proliferation, it cannot replicate and proliferate when introduced into the target cell, but it is able to express a large amount of αA-crystallin protein under the control of CMV promoter.

Pericyte loss is the main cause of blood-retinal barrier breakdown in early diabetes, and causes blindness in late diabetes. In this regard, the present inventors found that αA-crystallin disappeared in the pericytes of early diabetes. This protein is known to affect protection of the hyaloids vasculature and lens, but there is no report on a relationship between αA-crystallin and damaged pericytes in diabetes. Therefore, the present inventors investigated that the αA-crystallin reduction induced by diabetes in the retina contributes to apoptosis and loss of vascular endothelial cells and vascular leakage, and consequent BRB breakdown.

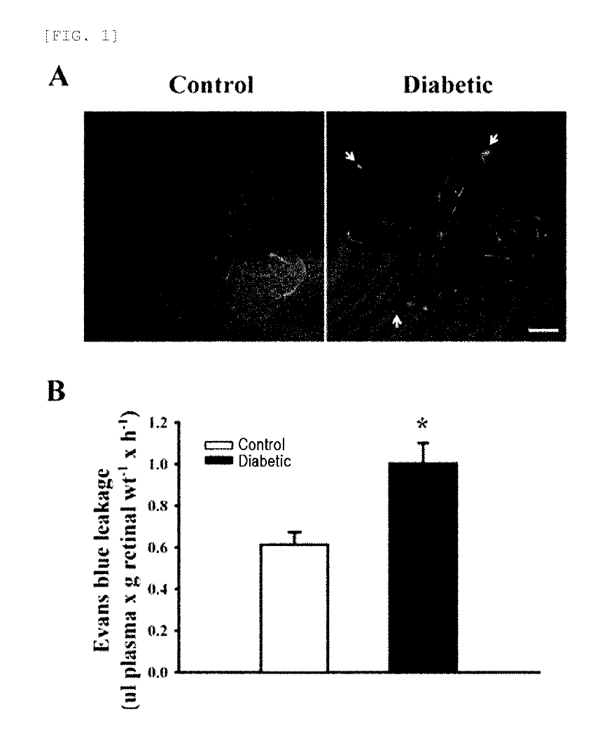

First, the increase of vessel leakage, cleaved PARP, active caspase-3, and pericyte apoptosis were observed in retinas of 2-month diabetic mice, compared with non-diabetic control mice (see FIG. 1). These results prove that pericyte loss and BRB breakdown are characteristic features in early diabetic retinopathy.

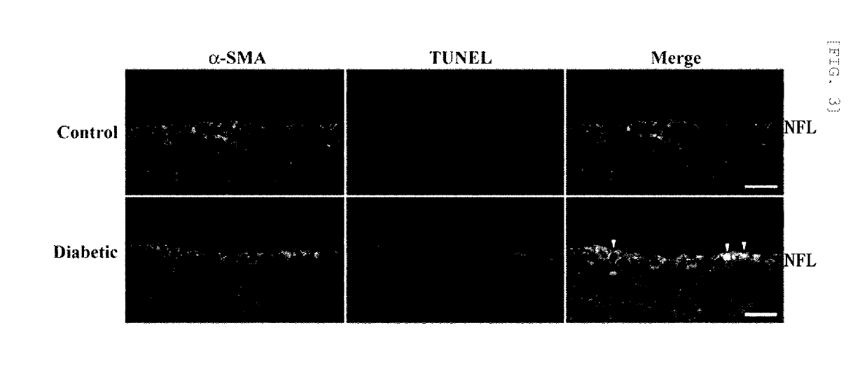

Proteomic analysis of changes in gene expression in retinas of 2-month diabetic mice and in the retinas of non-diabetic control mice confirmed significant decreases in two αA-crystallin subtypes in diabetic retinas (see FIG. 2). Interestingly, it was observed that αA-crystallin specifically reacted with α-SMA-positive pericytes in the nerve fiber layer of the retina (see FIG. 2D). This result indicates that αA-crystallin can be an important gene in control of the retinal pericytes in diabetes. Therefore, to verify the effects of αA-crystallin on vascular pathologies in the diabetic retina, the present inventors constructed a αA-crysta11in-containing recombinant adenovirus rAd-αAC-GFP and a control recombinant adenovirus rAd-GFP as adenovirus-mediated gene delivery systems (see FIG. 2A). Each of the recombinant adenoviruses was injected into the left and right vitreous of mice to examine changes in pericyte and vessel leakage in diabetic retinas. First, the results of GFP fluorescence imaging showed that adenovirus-mediated gene expression maintained in the retina vessels throughout the entire 10 weeks after adenovirus injections (see FIG. 3C), and such GFP expression indicates normal construction of the recombinant adenovirus (see FIG. 3E). It was also confirmed that the reduction of αA-crystallin in diabetic retinas is significantly compensated by treatment of αA-crystallin-containing recombinant adenovirus rAd-αAC-GFP.

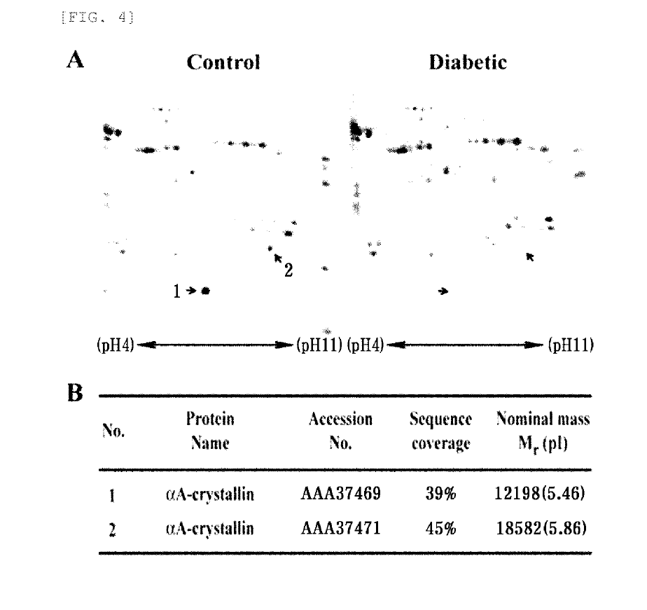

Next, it was confirmed that treatment of αA-crystallin-containing recombinant adenovirus rAd-αAC-GFP according to the present invention significantly inhibited diabetes-induced PARP cleavage and caspase-3 activation (see FIGS. 4B and 4C). Consistent with these results, the number of pericytes in retinal capillaries (0.01 mm²) was decreased in 2-month diabetic mice, compared with the control, but this decrease was effectively protected by treatment of the recombinant adenovirus rAd-αAC-GFP (see FIG. 4F). Consequently, it can be seen that αA-crystallin is present in retinal pericytes, and overexpression of αA-crystallin protects pericytes from their diabetes-mediated apoptosis.

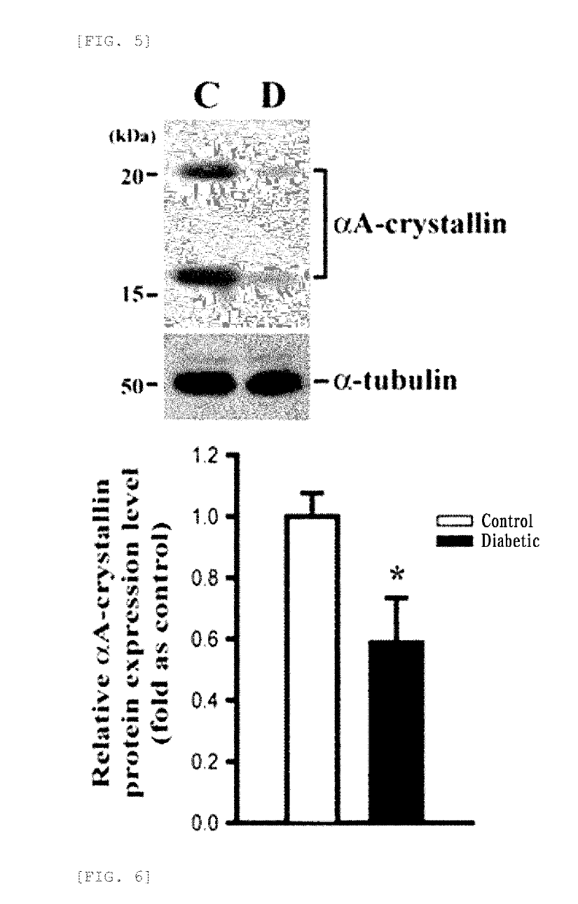

As pericyte loss is directly associated with vessel leakage, the effect of adenovirus on vessel leakage in diabetic retinas was examined. As a result, the diabetes-mediated increase in vessel leakage was effectively blocked with treatment of the αA-crystallin-containing recombinant adenovirus rAd-αAC-GFP according to the present invention (see FIG. 5). These results indicate that the αA-crystallin-expressing recombinant adenovirus can effectively protect the retina of diabetic mice from BRB breakdown caused by pericyte loss.

Taken together, overexpression of αA-crystallin through transferring adenovirus protects pericytes from diabetes-mediated natural cell death, thereby reducing BRB breakdown. Accordingly, the present inventors suggest that αA-crystallin present in pericytes can be a new therapeutic target for retinal vascular diseases including early diabetes.

In another aspect, the present invention provides a pharmaceutical composition for prevention or treatment of retinal vascular disease, comprising the recombinant adenovirus expressing an αA-crystallin gene as an active ingredient.

As used herein, the term, “prevention” refers to all of the actions by which the occurrence of retinal vascular diseases are restrained or retarded by administration of the pharmaceutical composition of the present invention. As used herein, the term “treatment” refers to all of the actions by which the symptoms of the diseases have taken a turn for the better or been modified favorably by administration of the pharmaceutical composition of the present invention.

As used herein, the term “retinal vascular disease” means all diseases caused by retinal vascular disorders in the eye, and examples thereof may include age-related macular degeneration (ARMD), choroidal neovascularization (CNV), retinopathy such as diabetic retinopathy, vitreoretinopathy, retinopathy of prematurity, glaucoma or the like, but are not limited thereto.

The pharmaceutical composition of the present invention may comprise the αA-crystallin-containing recombinant adenovirus as an active ingredient in a pharmaceutically effective amount, and further comprise a pharmaceutically acceptable carrier. As used herein, the term “pharmaceutically effective amount” means an amount sufficient to suppress or ameliorate the disease with a reasonable benefit/risk ratio applicable to the medical use, and the effective dosage level may be determined by those skilled in the art, depending on a patient's sex, age, body weight, health condition, the type and severity of disease, drug activity, sensitivity to the drug, administration method, time, and route, and excretion rate, treatment duration, factors including drugs used in combination or simultaneously, and other factors well known in the medical field.

The pharmaceutically acceptable carrier used in the pharmaceutical composition of the present invention may comprise, those typically used in formulation, lactose, dextrose, sucrose, sorbitol, mannitol, starch, acacia gum, calcium phosphate, alginate, gelatin, calcium silicate, microcrystalline cellulose, polyvinyl pyrrolidone, cellulose, water, syrup, methyl cellulose, methylhydroxybenzoate, propylhydroxybenzoate, talc, magnesium stearate, mineral oil or the like, but not limited thereto. In addition to the above ingredients, the pharmaceutical composition of the present invention may further comprise a lubricant, a wetting agent, a sweetener, a flavor, an emulsifier, a suspending agent, a preservative, or the like.

The pharmaceutical composition of the present invention may be administered via the route typically used in gene therapy, and parenteral administration is preferred, for example, intravenous administration, intraperitoneal administration, intramuscular administration, subcutaneous administration or local administration may be performed. The pharmaceutical composition of the present invention may be administered singly or in combination with other therapeutic agents, and may be administered with the conventional therapeutic agent serially or simultaneously.

The suitable dosage of the pharmaceutical composition of the present invention will depend upon a variety of factors including formulation method, the administration mode, the patient's age, body weight, sex, severity and symptoms of disease, diet, time and route of administration, the excretion rate, and reaction sensitivity. A physician having ordinary skill in the art may readily determine and prescribe the effective amount for desired treatment. In general, the pharmaceutical composition of the present invention may comprise the recombinant adenovirus of 2×10⁹ to 1×10¹⁰ pfu/ml. Typically, the recombinant adenovirus is injected in an amount of 2×10⁶ pfu every other day for 2 weeks.

The pharmaceutical composition comprising the recombinant adenovirus of the present invention may be prepared into a unit dosage form, or multiple dosage form along with a pharmaceutically acceptable carrier and/or excipient according to a method that can be easily employed by those skilled in the art. In this regard, the formulation may be in the form of solution in oily or aqueous medium, suspension, emulsion, extract, powder, granule, tablet or capsule, and may further comprise a dispersant or stabilizer.

In order to increase stability at room temperature, reduce the need for high-cost storage at low temperature, and prolong shelf-life, the pharmaceutical composition comprising the recombinant adenovirus of the present invention may be lyophilized. A process for freeze-drying may comprise the successive steps of freezing, first drying and second drying. The second drying process after freezing is to heat the composition under pressure to evaporate vapor. At the second drying step, absorbed residual water is removed from the dry product.

The freeze-dried formulation may comprise an excipient and a lyoprotectant. Non-limiting example of the excipient comprises a 0.9% buffer solution. The lyoprotectant functions to protect biological molecules during the freeze-drying, and supply mechanical support to the final product, which is exemplified by PBS (pH 7.0), and PBS/4%, 12% or 15% trehalose.

In still another aspect, the present invention provides a method for preventing or treating retinal vascular disease of a subject, comprising the step of administering the pharmaceutical composition comprising the recombinant adenovirus as an active ingredient to the subject.

As used herein, the term “subject” means all animals including human having the retinal vascular disease or being at the risk of having the disease, and the disease can be effectively prevented or treated by administering the pharmaceutical composition of the present invention to the subject. The pharmaceutical composition of the present invention may be administered in combination with the conventional therapeutic agent.

As used herein, the term “administration” means introduction of a predetermined substance into a patient by a certain suitable method. The composition of the present invention may be administered via any of the common routes, as long as it is able to reach a desired tissue. A variety of modes of administration are contemplated, including intraperitoneally, intravenously, intramuscularly, subcutaneously, intradermally, orally, topically, intranasally, intrapulmonarily and intrarectally, but the present invention is not limited to these exemplified modes of administration. A solid preparation for oral administration includes a tablet, a pill, a powder, a granule, a capsule, etc., and such a solid preparation may be prepared by mixing with at least one excipient, for example, starch, calcium carbonate, sucrose, lactose, gelatin, etc., in addition to the above composition. Also, in addition to the excipient, lubricants, such as magnesium stearate and talc, may be used. A liquid preparation for oral administration includes a suspension, a liquid for internal use, an emulsion, a syrup, etc., and in addition to a frequently used main diluent, such as water and liquid paraffin, the preparation may include a variety of excipients, for example, a wetting agent, a sweetening agent, an aromatic agent, a preservative, etc. However, since peptides are easily digested upon oral administration, active ingredients of a composition for oral administration should be coated or formulated for protection against degradation in the stomach. A preparation for parenteral administration includes a sterile aqueous solution, a non-aqueous solvent, a suspension, an emulsion, a freeze-dried preparation, and a suppository. As the non-aqueous solvent, and the suspension, propylene glycol, polyethylene glycol, vegetable oil such as olive oil, injectable ester such as ethyl oleate or the like may be used. As a base for the suppository, witepsol, macrogol, tween 61, cacao butter, laurin butter, glycerogelatin or the like may be used. To increase the stability or absorption of peptides, carbohydrates such as glucose, sucrose or dextran, antioxidants such as ascorbic acid or glutathione, chelating agents, low-molecular weight proteins or other stabilisers may be used.

Also, the pharmaceutical composition of the present invention may be administered with the aid of any means for delivering the active ingredient into target cells. Preferred administration mode and formulation are intravenous injection, subcutaneous injection, intracutaneous injection, intramuscular injection, instillation or the like. Injectable formulations may be prepared using an aqueous solvent, such as physiological saline, Ringer's solution or the like, or a non-aqueous solvent, such as vegetable oil, higher fatty acid ester (e.g., ethyl oleate, etc.), alcohol (e.g., ethanol, benzyl alcohol, propylene glycol or glycerin) or the like, and may comprise a pharmaceutically acceptable vehicle, such as an antiseptic stabilizer (e.g., ascorbic acid, sodium hydrogen sulfite, sodium pyrosulfite, BHA, tocopherol, EDTA, etc.), an emulsifier, a pH-adjusting buffer, an anti-microbial preservative (e.g., phenylmercuric nitrate, thimerosal, benzalkonium chloride, phenol, cresol, benzyl alcohol, etc.) or the like.

Hereinafter, the present invention will be described in more detail with reference to Examples. However, these Examples are for illustrative purposes only, and the invention is not intended to be limited by these Examples.

### EXPERIMENTAL EXAMPLE 1

### Animal Experiment

Male C57BL/6 mice (KOATEC), weighing 20 to 22 g (8-week-old), were maintained on a standard rodent diet and water ad libitum, and handled in strict accordance with the Institutional Animal Care and Use Committee of Gyeongsang National University. To induce diabetes, intraperitoneal injection of 55 mg/kg STZ (streptozotocin; USA, Mo., St Louis, Sigma) in 50 mmol/L sodium citrate (pH 4.5) into the mice was performed once every five days. Sex·age-matched control mice received buffer alone. All mice were used for experiments after 2 months. Blood samples were collected by tail puncture after a 2-hour fast, and blood glucose levels were measured using a glucometer (UK, Precision). Diabetes was defined as blood glucose levels >13.9 mmol/L at 1 week after five-time STZ injections.

### EXPERIMENTAL EXAMPLE 2

### Antibody and Assay Kit

For detection of cell death, a TUNEL (terminal deoxynucleotidyl transferase (TdT)-mediated dUTP nick-end labeling) assay kit and TMR red (In Situ cell death detection kit, Germany, Mannheim, Roche) were used. For Western blotting, an ECL (enhanced chemiluminescent, Amersham Biosciences) kit was used. At this time, mouse monoclonal antibodies against αA-crystallin, α-smooth muscle actin (α-SMA; marker for pericytes), and α-tubulin (respectively from Santa Cruz Biotechnologies, Chemicon, and Sigma); rabbit polyclonal antibodies against αA-crystallin, active caspase-3 and GFP (all from Abcam); and the rabbit polyclonal PARP (poly-ADP-ribose-polymerase) antibody (from Cell signaling) were used as primary antibodies, and HRP (horseradish peroxidase)-conjugated anti-mouse and anti-rabbit IgGs (Pierce) were used as secondary antibodies, Alexa Fluor™ 488 and 594 goat anti-mouse IgG, and Alexa Fluor 488 goat anti-rabbit IgG (Invitrogen) were also used for immunofluorescent staining.

### EXPERIMENTAL EXAMPLE 3

### Construction of a Recombinant Adenovirus Expressing αA-Crystallin

A sequence encoding mouse αA-crystallin was amplified by PGR, and cloned into the entry vector, followed by confirming sequence. A template of αA-crystallin used in PGR was cDNA clone MGC:115743 (openbiosystems), and two primers of SEQ ID NOs. 1 and 2 and pfu tag polymerase were used to perform amplification for 15 cycles consisting of at 94° C. for 1 minute, at 56° C. for 1 minute and at 72° C. for 1 minute. An entry vector inserted with an αA-crystallin gene was constructed using a Gateway system (Invitrogen), and sequencing was performed to confirm whether the gene identical to the template was amplified. Further, in order to confirm the expression position of αA-crystallin, green fluorescent protein was amplified by PCR, and inserted at its N-terminal. In order to construct αA-crystallin-containing recombinant adenovirus vector, an adenovirus expression system (ViraPower™ Adenoviral Expression System, Invitrogen) was used. The full length DNAs of mouse αA-crystallin and green fluorescent protein (GFP) were simultaneously inserted into a gateway-based pAd/CMV/V5-DEST vector (Invitrogen). Recombinant adenoviral DNAs (pAD-αA-crystallin-GFP) were transfected into 293A cells (Invitrogen) using a Lipofectamine 2000 reagent (Invitrogen). In order to prepare a GFP-containing recombinant adenovirus used as a control group, a pAd-GFP vector (Invitrogen) was digested with a restriction enzyme Pacl, and transfected into 293A cells. Viral titers were determined using a PFA (plaque-forming assay) on 293 cells. FIG, 7A shows structures of αA-crystallin-GFP-expressing recombinant adenovirus (rAd-αAC-GFP) and GFP-expressing recombinant adenovirus (rAd-GFP) thus prepared. The prepared recombinant adenovirus was amplified in 293A cells, and purified using a purification kit (Adeno-XTM Virus Purification Kit, Calif., Mountain View, Clontech Laboratories, Inc.). Consequently, a recombinant adenovirus rAD-αAC-GFP comprising a nucleic acid sequence encoding a mouse-derived wild-type αA-crystallin protein, a CMV promoter, a GFP reporter gene and a polyadenylation signal was prepared (FIG. 4A). This recombinant adenovirus was deposited at the gene bank (KCTC) of Korea Research Institute of Bioscience and Biotechnology (KRIBB) on Jan. 6, 2011, and endowed with the deposition number of KCTC 11844BP.

### EXPERIMENTAL EXAMPLE 4

### Treatment of Recombinant Adenovirus

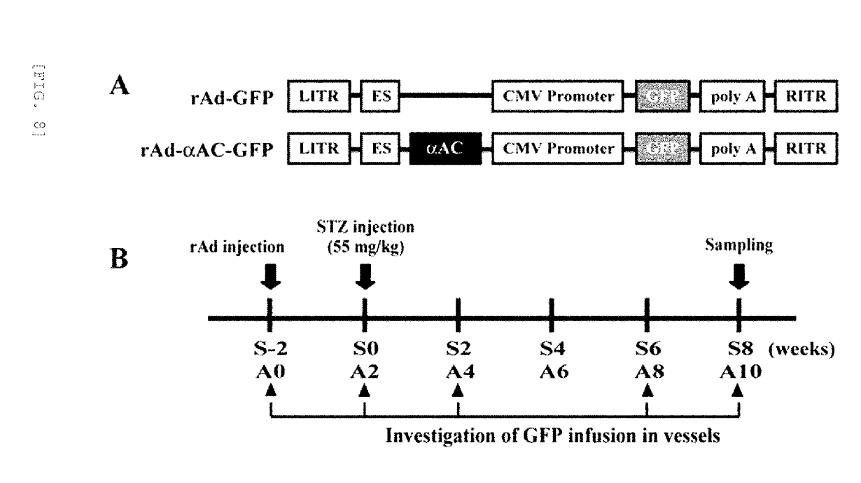

Mice were anesthetized by intramuscular injections of 50 mg/kg of zoletil 50 (125 mg of tiletamine and 125 mg of zolasepam) (France, Carros, Virbac Laboratories) in the quadriceps muscles of thigh. The eye was topically anesthetized with 4% araethocaine drops, and the pupils were dilated with 1% tropicamide and 2.5% phenylephrine hydrochloride drops. Each 2 μl of αA-orystallin-GFP (rAd-αAC-GFP; 2×10⁸ pfu/eye) and GFP (rAd-GFP; 1×10⁹ pfu/eye)-expressing recombinant adenovirus were injected with a 30-gauge needle into the left and right vitreous of mice under an operating microscope. At 2 weeks after the recombinant adenovirus injection, diabetes was induced with STZ injection. The control group was injected with buffer. The testing efficiency of the recombinant adenovirus was confirmed via GFP fluorescence imaging in retinal vessels at 0, 2, 4, 8, and 10 weeks after injection of the virus. Experiments were carried out at 2 months after diabetes induction, that is, at 10 weeks after injection of the virus. A schematic diagram of the animal model and the adenovirus treatment is shown in FIG. 7B.

### EXPERIMENTAL EXAMPLE 5

### Visualization of Retinal Vasculature

Retinal vasculature was assessed by angiography using FITC-D (high-molecular-weight fluorescein isothiocyanate-conjugated dextran; MW 2×10⁶, Sigma) and TMR-D (tetramethylrhodamine dextran; MW 2×10⁶, Invitrogen). Mice were anesthetized with zoletil, and the descending aorta was clamped. FITC-D and TMR-D were dissolved in 0.05 M sodium citrate (pH 4.5) at final concentrations of 50 mg/ml and 10 mg/ml, respectively, and 1 ml of FITC-D and 0.5 ml of TMR-D were infused through the left ventricle of each mouse. All mice were sacrificed immediately after dye injection, and the retinas were carefully dissected and fixed in fresh 4% paraformaldehyde (PFA) for 6 hours, and washed with 0.01 M PBS (phosphate-buffered saline). Subsequently, the retinas were dissected by radial incisions without damaging retinal blood supply, and then flat-mounted with a ProLong Gold anti-fade reagent (Invitrogen).

### EXPERIMENTAL EXAMPLE 6

### Measurement of BRB Breakdown

BRB breakdown was evaluated using an Evans Blue leakage assay as described previously (Kim Y H et al., Life Sci. 2007; 31(14): 1167-1173). Mice were anesthetized, and then received injections into the left jugular vein of 45 mg/kg Evans Blue dye. And the circulation was maintained for 2 hrs. After injection, 0.2 ml of blood was extracted through the left ventricle, and mice were perfused with 37° C. normal saline to completely remove the Evans Blue dye in blood vessels. Retinas were carefully dissected and completely dried using a Speed-Vac for 5 hours. The dry weight was measured. Then, retinas were incubated in 120 ml of formamide (Sigma) for 18 hours at 70° C. to extract the Evans Blue dye, and the extract was filtered through an ultra-filter (30,000 MW). The supernatant was used for spectrophotometry three times, and each measurement was performed at 5-second intervals. Absorbance of each sample was measured at 620 nm. The Evans Blue dye concentration in retina extracts was calculated using a standard curve of Evans Blue in formamide and normalized to the weight of dried retina, and expressed as μl plasma×g dried, retina wt⁻¹×h⁻¹.

### EXPERIMENTAL EXAMPLE 7

### Immunofluorescence Staining

Preparation of retinal cryosections and immunohistostaining were performed in accordance with the method as described previously (Kim Y H et al., Diabetes. 2008; 57(8): 2181-2190). Mice were anesthetized, and the retinas were immediately excised from the eyes. The retinas were immersed in 4% PFA for 6 hours, and washed with PBS. Then, the retinas were sequentially immersed in 10% and 20% sucrose for 2 hours, and then immersed in 30% sucrose at 4° C. overnight. The retina tissues were frozen using O.C.T. compound (Japan, Tokyo, Sakura) in liquid nitrogen, and retinal sections with 12-μm thickness were prepared using a freezing microtome (Japan, Tokyo, Leica, Leica 8400E). The sections were washed with 0.01 M Tris-buffered saline, and fixed in 4% PFA at room temperature for 20 minutes, and then washed. Subsequently, the sections were washed with 0.05% Tris-triton X-100 on ice for 5 minutes, and blocked with a signal enhancer for 30 minutes, followed by incubation with rabbit anti-αA-crystallin (1:200; abcam) and mouse anti-α-SMA (1:200; Chemicon) antibodies at 4° C. overnight. After washing with Tris, sections were reacted with Alexa-Fluor antibody (Invitrogen) for 1 hour and mounted using an anti-fade reagent. In order to examine immunoreactivity between αA-crystallin and α-SMA, sections were sequentially incubated with the signal enhancer, primary antibody mixture, and Alexa Fluor 488 and 594 goat anti-rabbit and -mouse IgGs mixture.

### EXPERIMENTAL EXAMPLE 8

### Measurement of Pericytes

In order to examine pericytes surrounding retinal vessels, immunofluorescence staining of angiography with infusion of TMR-D (Invitrogen) was performed to dye the blood vessels and pericytes with the pericyte marker α-SMA, as described above. Subsequently, mice were completely anesthetized, and the descending aorta was carefully clamped. Then, 0.5 ml of TMR-D at a final concentration of 10 mg/ml was infused through the left ventricle of each mouse. After infusion, whole retina flats were carefully prepared, as described above, and immunofluorescence staining for α-SMA was performed. TMR-D-stained retinas were sequentially incubated with the signal enhancer for 10 minutes, α-SMA antibody at 4° C. overnight, and Alexa Fluor 488 goat anti-mouse IgG (Invitrogen) for 1 hour, and mounted using the anti-fade reagent. All images were observed under a confocal microscope, and the number of vascular endothelial cells in the fixed field (0.01 mm² vessel) in 4 different images of the whole retina of each group was counted.

### EXPERIMENTAL EXAMPLE 9

### Determination of Adherent Leukocytes

In order to evaluate retinal leukocyte adhesion, the ex-vivo retinal leukostasis was induced by the method described previously with some modifications (Joussen A M et al., FASEB J. 2004; 18(12): 1450-1452). After anaesthetizing mice, the chest cavity was opened and the descending aorta was carefully clamped. To remove erythrocytes and non-adherent leukocytes, 1.5 ml/g mouse body weight of 1×PBS was perfused into the left ventricle. Fixation was achieved by perfusion with 20 ml of 1% PFA under physiological pressure. Non-specific binding was blocked with 1% BSA (bovine serum albumin) in PBS, followed by perfusion with TRITC (TMR-isothiocyanate)-coupled concanavalin A lectin (20 μg/ml in PBS, pH 7.4; 5 mg/kg BW; vector Labs, Burlingame, Calif.) to label adherent leukocytes and vascular endothelial cells. At 5 minutes after perfusion, to remove residual unbound lectin, 2 ml of PBS and 1% BSA/PBS were perfused sequentially. The whole retinas were carefully dissected, and then flat-mounted with the anti-fade reagent. Subsequently, the total numbers of leukocytes in the retinal arteriole, veinlet and capillary were counted.

### EXPERIMENTAL EXAMPLE 10

### TUNEL Analysis

In order to examine death of α-SMA- and αA-crystallin-positive cells, cryosections were sequentially subjected to a TUNEL assay using TMR red and immunofluorescent staining for αA-crystallin and α-SMA in accordance with the manufacturer's instructions as described previously (Kim Y H et al., Diabetes. 2010; 59(7): 1825-1835). The sections were fixed in 10% formaldehyde at room temperature for 20 minutes, and washed with 0.1 M Tris for 3 minutes three times, and then incubated in a blocking solution (3% H₂O₂ dissolved in methanol) for 10 minutes. Subsequently, the sections were permeated with 0.1% sodium citrate in Tris buffer supplemented with 0.1% triton X-110 on ice for 5 minutes, and washed with Tris. The fixed and permeated sections were covered with parafilm and incubated in a TUNEL reaction mixture at 37° C. for 1 hour. After washing with Tris, immunofluorescent staining for α-SMA and αA-crystallin was performed in accordance with the above described method. The retina sections were sequentially incubated with the signal enhancer, primary antibody against α-SMA and αA-crystallin, and Alexa Fluor 488 goat anti-mouse IgG and anti-rabbit IgG. All reactions were performed in a wet chamber in order to prevent evaporation loss. The experiment was repeated for 4 different retinas of each group.

### EXPERIMENTAL EXAMPLE 11

### Western Blot Analysis

Total retinal proteins were extracted from 4 different retinas of each group and Western blot analysis was performed as described previously (Kim Y H et al., Neurobiol Dis 2007; 28(3): 293-303). The total proteins (30 μg) obtained from the retinas were analyzed by 10% SDS-PAGE, and transferred onto a nitrocellulose membrane. The blot was blocked in 3% BSA-containing 0.01 M Tris-0.5% Tween 20 (TBS-T) at 4° C. overnight, and incubated with αA-crystallin, PARP, and active caspase-3 antibody (1:4,000, 1:1,000, 1:500; Santa cruze, Cell signaling, abcam), and HRP-conjugated goat anti-mouse and rabbit IgGs (1:10,000) for 2 hours and 1 hour, respectively. The immunoreactive proteins were visualized using an ECL (enhanced chemiluminescent) kit (USA, NJ, Amersham Biosciences, Piscataway). The blot was stripped, and reblotted with α-tubulin antibody (1:50,000; Sigma) as a loading control group. The same proteins were compared with the control group that was normalized to α-tubulin content, and the fold change in the protein level was represented in a bar graph.

### EXPERIMENTAL EXAMPLE 12

### Two-Dimensional Gel Electrophoresis (2-DE) and Protein Identification

2-DE was performed as described previously (Kim Y H et al., Neurobiol Dis 2007; 28(3): 293-303). The total protein from each retina (100 μg/strip) was resuspended in rehydration buffer [2 M thiourea/6 M urea, 4% CHAPS, 65 mM DTT, 0.5% ampholytes, 0.002% bromophenol blue (BPB)], loaded onto 7-cm IPG strips (Immobiline DryStrip™, pH4-11 linear gradient, Genomine Inc.), and incubated in IEF (isoelectric focusing) tray (Bio-rad) overnight. IEF was performed at 250 V for the first 15 minute for sample injection, followed by a gradient increase from 4,000 to 10,000 VH. IPG strips were equilibrated in an equilibration buffer I (6 M urea, 2% SDS, 30% glycerol, 0.002% BPB, 50 mM Tris-HCl, pH 8.8, 2% DTT) and in an equilibration buffer II [6 M urea, 2% SDS, 30% glycerol, 0.002% BPB, 50 mM Tris-HCl, pH 8.8, 2.5% iodine acetamide (IAA)] for 15 minutes, respectively. After IEF and equilibration, IPG strips were subjected to 13.5% SDS-PAGE. Protein spots were visualized using silver staining. In short, a gel was fixed in a solution containing 50% methanol, 12% acetic acid, and 0.5 ml of 37% formaldehyde for 2 hours. All incubations were performed in a shaker under gentle agitation. After fixation, the gel was washed with 50% ethanol for 20 minutes twice, and then treated in advance with Na₂S₂.5H₂O (0.1 g/l) for 1 minute, and washed with secondary distilled water. The gel was incubated in AgNO₃ (2 g/l) and 0.75 ml of 37% formic acid for 30 minutes to infiltrate silver into the gel. Then, the gel was washed with secondary distilled water for 20 seconds three times. A color development solution containing Na₂CO₃ (60 g/l), Na₂S₂.5H₂O (4 mg/l), and 0.5 ml of 37% formic acid was prepared in advance, and stored in ice slurry. For visualization, the gel was incubated in the color development solution, until clear images were developed. When clear spots appeared, the gel was washed with secondary distilled water for 20 seconds, and 50% methanol and 12% acetic acid were added for 10 minutes to terminate the reaction. Proteins were identified by PMF (peptide mass fingerprinting, Genomine Inc.) analysis using MALDI-TOF mass spectrometry (Ettan MALDI Pro). Gel staining, image analysis, and protein identification were performed once for 4 different retinal extracts of each group.

### EXPERIMENTAL EXAMPLE 13

### Image Capture and Statistical Analysis

All retinal images were obtained with an IX2-DSU disk-scanning microscope at 20×, 40×, 400×, and 600× magnifications (Olympus, Wendenstrasse, Hamburg, Germany), and a confocal microscope (Axioplan2 Imaging, Zeiss). Images of the retinal sections were captured at a distance of approximately 0.8 to 1 mm from the optic nerve head. Data were analyzed with the Soft Imaging System (Jandel Scientific, Erkrath, Germany) and SigmaGel 1.0 software, and all calculations were performed using SigmaPlot 4.0 (SPSS Inc., Chicago, Ill., USA) software. Statistical analyses were performed using the Kruskal-Wallis H test and Mann-Whitney U test (SPSS). Results are presented as means±SEM from four independent values, and considered significant at p<0.05.

### EXAMPLE 1

### Induction of Vascular Leakage, Apoptotic Molecule, and Pericytes Apoptosis in Retinas by Diabetes

The results of fluorescent angiography showed diabetes-induced vessel leakage (arrows) in the peripheral retinas, but not in control retinas (FIG. 1A). In addition, an Evans Blue leakage assay showed a 1.63-fold increase in vessel leakage in diabetic retinas, compared with controls (0.61±0.061 vs. 1.0023±0.099; P=0.0046, n=10) (FIG. 1B). Western blot analysis for apoptosis showed 1.55˜1.57-fold or more increase in cleaved PARP and active caspase-3 levels in diabetic retinas, compared with controls (1±0.060 vs. 1.545±0.147 and 1±0.122 vs. 1.573±0.218; P=0.001 and 0.045, n=6, respectively) (FIG. 2). Double immunofluorescence staining for TUNEL and the pericyte marker α-SMA revealed an increase in TUNEL-positive pericytes (arrowheads) in diabetic retinas, compared with controls (FIG. 3).

As shown in FIGS. 1 to 3, vessel leakage, cleaved PARP, active caspase-3, and pericyte apoptosis were increased in retinas of 2-month diabetic mice, indicating that the diabetic animal model of the present invention showed pericyte apoptosis and BRB breakdown as the characteristics of early diabetic retinopathy.

### EXAMPLE 2

### Suppression of αA-Crystallin Expression in Retinal Pericytes Induced by Diabetes

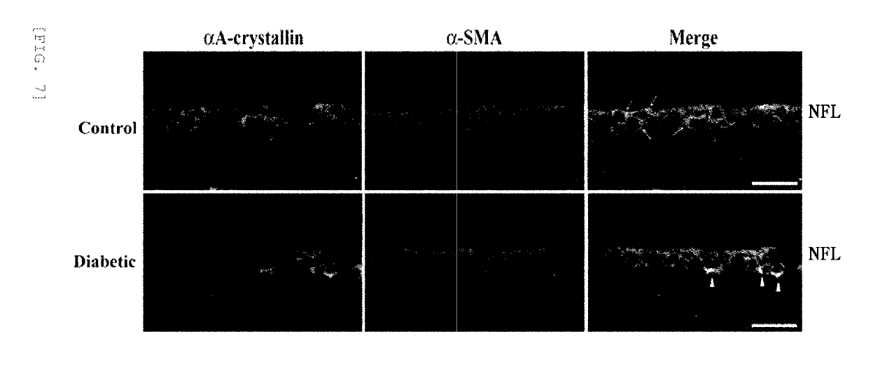

To investigate changes in gene expression in the control retina and in retinas of 2-month diabetic mice, proteomic analysis was performed using total retinal proteins. The results of 2-DE silver staining showed disappearance of two spots in diabetic retinas, compared with controls (1 and 2 in FIG. 4A), and the results of PMF analysis using MS-MALDI-TOF showed that these spots were two αA-crystallin subtypes (FIG. 4B). The results of Western blot analysis of αA-crystallin showed the two bands (17 and 20 kDa) of αA-crystallin, and the total amount of both αA-crystallin isoforms was decreased by approximately 40% in diabetic retinas, compared with controls (1±0.077 vs. 0.587±0.147; P=0.0322, n=6) (FIG. 5). Double immunofluorescence staining for αA-crystallin and α-SMA revealed predominant levels of αA-crystallin in retinal pericytes in both control and diabetic mice (arrows and arrowheads in FIG. 6).

As shown in FIGS. 4 to 6, proteomic assessment of changes in gene expression in retinas of 2-month diabetic mice and control groups confirmed great decreases in two αA-crystallin subtypes in diabetic retinas. In addition, αA-crystallin immunoreactivity was specific to α-SMA-positive pericytes in the nerve fiber layer (NFL) of both retinas (FIG. 6), but the expression was not positive to ganglion cells or astrocytes (data not shown). Therefore, these results suggest that αA-crystallin is a main gene regulating the pericytes in diabetic retina.

### EXAMPLE 3

### Protective Effect of αA-Crystallin-Expressing Recombinant Adenovirus on αA-Crystallin Reduction in Diabetic Retina

To verify the effects of αA-crystallin on vascular pathology in the diabetic retina, adenovirus-mediated gene delivery systems were used. rAd-aAC-GFP and rAd-GFP were introduced into the left and right vitreous body of mouse, and the pericytes and vessel leakage were examined in the diabetic retinas, compared to the control group. At 2 weeks after injection, diabetes was induced by STZ injection. The retinal reactions were examined at 2 months after diabetes induction, that is, at 10 weeks after virus injection.

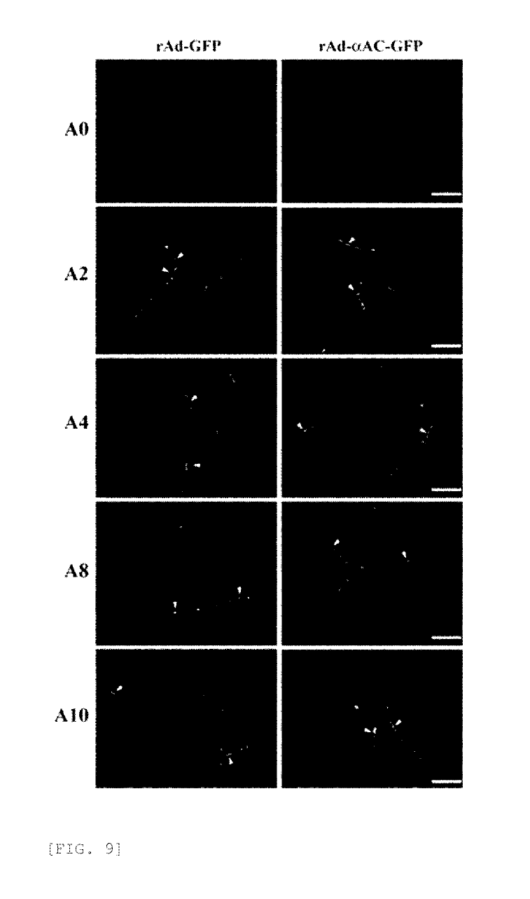

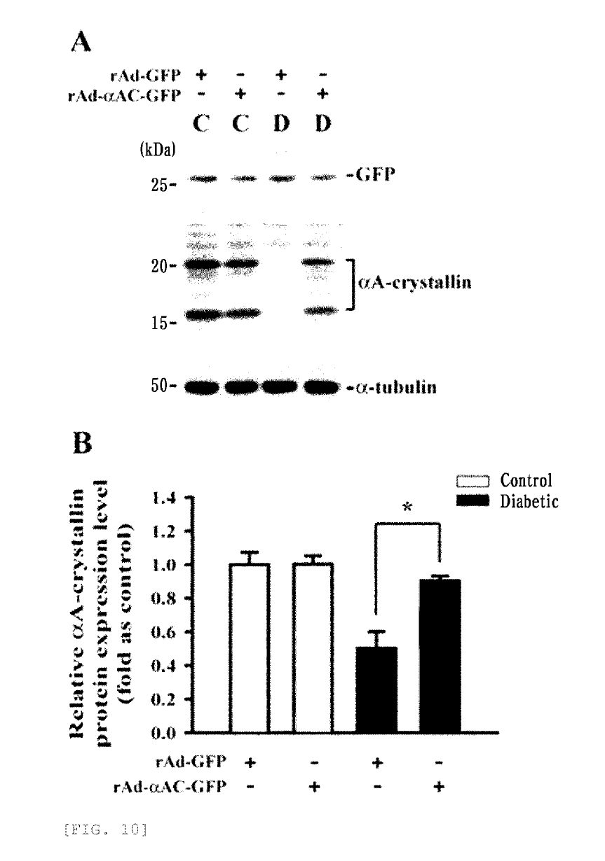

FIGS. 7A and 7B show schematic diagrams for constructs of the recombinant adenovirus expressing GFP (rAd-GFP, control) and the recombinant adenovirus expressing αA-crystallin and GFP (rAd-αAC-GFP) prepared in the present invention, and the treatment procedure of animal model with the recombinant adenoviruses. In order to examine permeation of the recombinant adenovirus into the retina, rAd-GFP and rAd-αAC-GFP were injected, and GFP fluorescence imaging was performed for the retinal vessels of normal mice at 0, 2, 4, 3, and 10 weeks. The results showed that GFP expression was maintained in the vessels for up to 10 weeks after virus injection (FIG. 8). FIGS. 9A and 9B showed that αA-crystallin protein reduction was significantly reduced in the diabetic retinas at 2 weeks by treatment of rAd-αAC-GFP, compared to rAd-GFP (0.502±0.099 vs 0.903±0.028; P=0.0046, n=5), while αA-crystallin levels were not affected in the control mouse. GFP expression was similarly induced in all retinas after injection of two types of virus (FIG. 9A).

As a result, GFP fluorescence imaging showed that adenovirus-mediated gene expression continued in the retinal vessels throughout the entire 10 weeks after adenovirus injections (FIG. 8). It was also confirmed that the rAd-αAC-GFP treatment significantly protected the retina from diabetes-induced αA-crystallin reduction, and GFP protein was ultimately expressed after injections of rAd-aAC-GFP and rAd-GFP in the retinas of the control and diabetic groups (FIG. 9B), indicating that the recombinant adenovirus constructed in the present invention functions desirably.

### EXAMPLE 4

### Protective Effect of αA-Crystallin-Expressing Recombinant Adenovirus on Diabetes-Induced Apoptotic Protein or Pericyte Loss

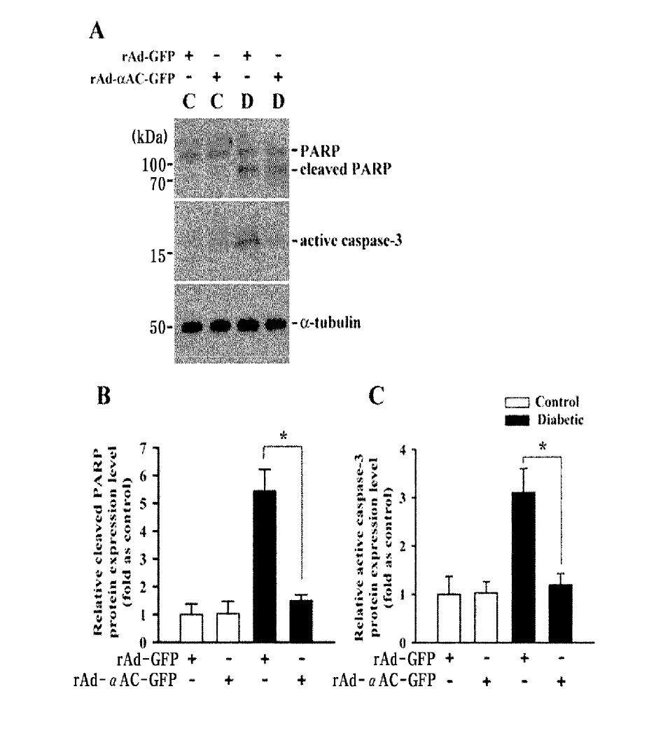

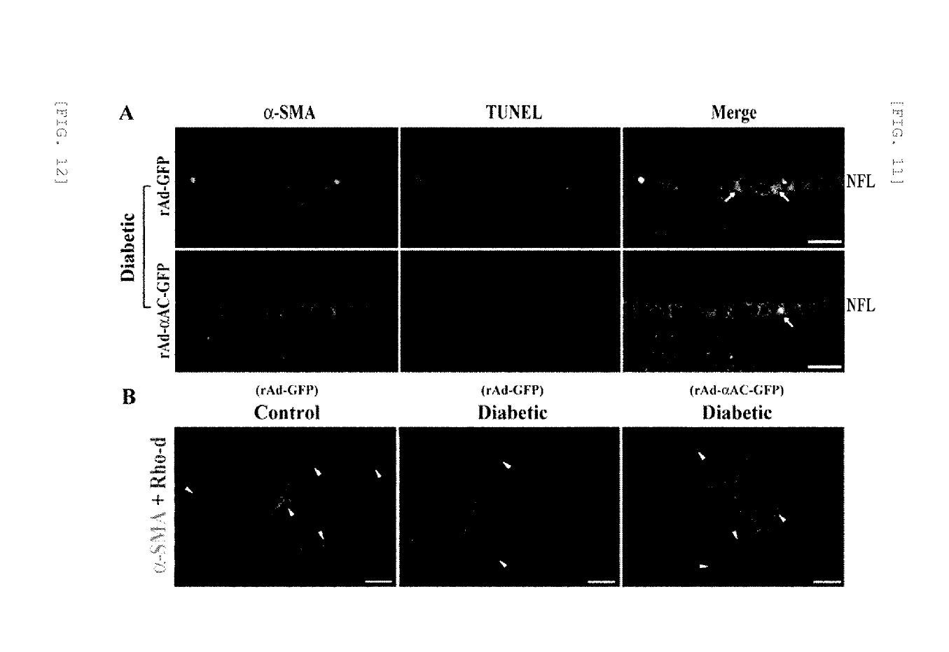

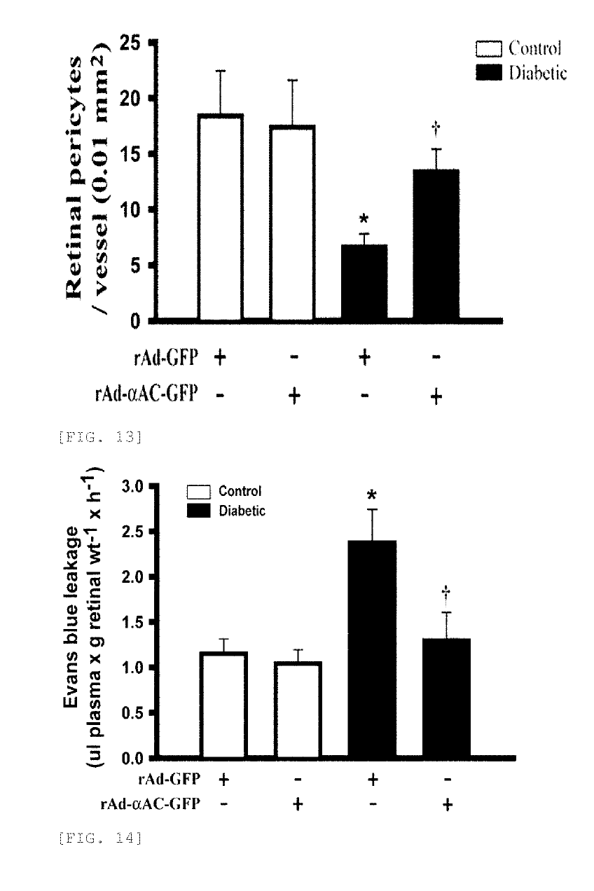

Western blot analysis of the cleaved PARP and active caspase-3 showed that the increased protein in retinas of 2-month diabetic mice were decreased to approximately 3 to 4-fold by treatment of rAd-aAC-GFP, compared to rAd-GFP (5.44±0.775 vs 1.495±207 and 3.104±0.497 vs 1.195±0.237; P=0.0027 and 0.0133, n=4, respectively) (FIGS. 10A to 10C). In particular, TUNEL-positive retinal pericytes decreased in rAd-aAC-GFP-treated diabetic mice: compared with those in rAd-GFP-treated diabetic mice (arrows of FIG. 11A). In addition, retinal pericytes surrounding retinal vessels (arrowheads) were observed by double immunofluorescence staining for the vessel and pericyte markers, TMR-D and a-SMA in both control and diabetic mice treated with adenovirus (FIG. 11B). The number of pericytes per vessel (0.01 mm²) decreased by 2.7-fold in 2-month diabetic mice, compared with the control (18.38±4.062 vs 6.64±1.176; P=0.024 , n=5), but the pericyte loss was effectively blocked in diabetic retinas by treatment of rAd-αAC-GFP, compared with rAd-GFP (6.64±1.176 vs 13.38±2.03; P=0.021, n=5) (FIG. 12). The rAd-αAC-GFP treatment did not affect the number of pericytes in the control mouse, and the images thereof were omitted in FIG. 11B.

The diabetes-mediated PARK cleavage and caspase-3 activation were greatly reduced by treatment of rAd-aAC-GFP, compared with rAd-GFP (FIGS. 10 to 12). In addition, TUNEL-positive retinal pericytes present in NFL decreased in rAd-aAC-GFP-treated diabetic mice, compared with those in rAd-GFP-treated diabetic mice. Consistent with these results, the number of pericytes in retinal capillary (0.01 mm²) decreased in 2-month diabetic mice, compared with the control group. However, this decrease was effectively protected by treatment of rAd-aAC-GFP (FIG. 12). Taken together, these results indicate that αA-crystallin exists in the retinal pericytes, and the overexpression of αA-crystallin protects the pericytes from diabetes-mediated cell death.

### EXAMPLE 5

### Protective Effect of αA-Crystallin-Expressing Recombinant Adenovirus on Diabetes-Induced Vessel Leakage

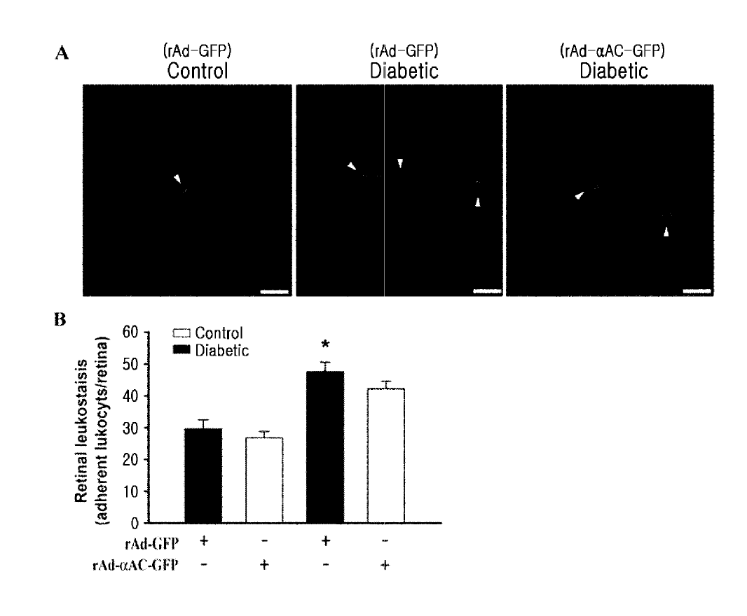

Pericyte loss is directly involved in vessel leakage, and thus the effect of the adenovirus of the present invention on the vessel leakage was examined in the diabetic retina. As expected from the above Examples, the increase in the diabetes-mediated vessel leakage was effectively blocked by treatment of rAd-aAC-GFP (FIGS. 13 and 14). These results indicate that the αA-crystallin-expressing adenovirus effectively protects BRB breakdown due to pericyte loss in diabetic retinas. Specifically, the results of Evans Blue leakage assay showed that although the diabetic retinas showed 2.67-fold hrgher BRB breakdown than the control retinas (0.955±0.245 vs 2.55±0.5034; P=0.018, n=4), BRB breakdown increased in the diabetic retina was significantly reduced by treatment of rAd-aAC-GFP, compared with rAd-GFP (2.55±0.5034 vs 1.29±0.222; P=0.046, n=6) (FIG. 13).

Meanwhile, leukocyte adhesion to the retinal vasculature is also one of the features of the early diabetic retinopathy, and BRB breakdown, endothelial cell injury and pericyte apoptosis are known. In the present invention, TRITC-coupled, concanavalin-A-stained adherent leukocytes (arrowheads) in vessels were counted in whole retinas (FIG. 14A). In addition, the adherent leukocytes were significantly increased in the entire retinas at 2 months after induction of diabetes, compared with controls, but leukocyte adhesion was not regulated by treatment of rAd-aAC-GFP (FIG. 14E). That is, leukocyte adhesion was increased by 1.6-fold in the diabetic retinas, compared with the control (29.6±2.839 vs 47.6±2.977; P=0.0011, n=5), but the number of adherent leukocytes was not significantly affected by rAd-aAC-GFP treatment, compared with rAd-GFP treatment (47.6±2.977 vs 42.2±2.417; P=0.197, n=5) (FIG. 14E).

These results indicate that leukocyte adhesion is not directly affected by αA-crystallin overexpression due to adenovirus transfer. Therefore, BRB breakdown mediated by αA-crystallin induction in retinal pericytes is separated from leukocyte adhesion.

Taken together, the present invention indicate that αA-crystallin overexpression by viral transfer plays an important role in early BRB breakdown in diabetes, which can be mediated by αA-crystallin-induced pericyte survival. Therefore, αA-crystallin in retinal pericytes may provide a new therapy for blood vessel abnormalities in early diabetes.

### Effect of the Invention

According to the present invention, gene therapy using the recombinant adenovirus expressing an αA-crystallin gene increases the expression level of the αA-crystallin gene in the damaged retinal pericytes to suppress pericyte death and loss, retinal vascular leakage, and leukocyte adhesion surrounding retinal vessels, thereby protecting the pericytes. Therefore, it can be used for the prevention and treatment of various retinal vascular diseases including diabetic retinopathy.

1. A recombinant adenovirus expressing an αA-crystallin gene.

2. The recombinant adenovirus according to claim 1, wherein the αA-crystallin gene has a nucleic acid sequence represented by SEQ ID NO. 4 (Genebank ID: BC085172).

3. The recombinant adenovirus according to claim 1, further comprising a promoter operably linked to the αA-crystallin gene, a polyadenylation signal sequence, and a reporter gene.

4. The recombinant adenovirus according to claim 1, wherein the recombinant adenovirus is rAd-αAC-GFP (deposition number: KCTC 11844BP).

5. A pharmaceutical composition for the prevention or treatment of retinal vascular disease, comprising the recombinant adenovirus of claim 1 expressing an αA-crystallin gene as an active ingredient.

6. The pharmaceutical composition according to claim 5, wherein the retinal vascular disease is selected from the group consisting of age-related macular degeneration (ARMD), choroidal neovascularization (CNV), diabetic retinopathy, vitreoretinopathy, retinopathy of prematurity, and glaucoma.

7. The pharmaceutical composition according to claim 5, further comprising a pharmaceutically acceptable carrier.

8. A method for preventing or treating retinal vascular disease of a subject, comprising the step of administering the composition of claim 5 to the subject.

9. The method according to claim 8, wherein the retinal vascular disease is selected from the group consisting of age-related macular degeneration (ARMD), choroidal neovascularization (CNV), diabetic retinopathy, vitreoretinopathy, retinopathy of prematurity, and glaucoma.

10. The pharmaceutical composition according to claim 5, wherein the αA-crystallin gene has a nucleic acid sequence represented by SEQ ID NO. 4 (Genebank ID: BC085172).

11. The pharmaceutical composition according to claim 5, wherein the recombinant adenovirus further comprises a promoter operably linked to the αA-crystallin gene, a polyadenylation signal sequence, and a reporter gene.

12. The pharmaceutical composition according to claim 5, wherein the recombinant adenovirus is rAd-αAC-GFP (deposition number: KCTC 11844BP).

13. The method according to claim 8, wherein the αA-crystallin gene has a nucleic acid sequence represented by SEQ ID NO. 4 (Genebank ID: BC085172).

14. The method according to claim 8, wherein the recombinant adenovirus further comprises a promoter operably linked to the αA-crystallin gene, a polyadenylation signal sequence, and a reporter gene.

15. The method according to claim 8, wherein the recombinant adenovirus is rAd-αAC-GFP (deposition number: KCTC 11844BP).


SEQUENCE LISTING (4 sequences)

SEQ ID NO: 1 - DNA, 54 bp, Artificial Sequence
  Forward primer for alpha A-crystallin
ggggacaagtttgtacaaaaaagcaggctccaccatggacgtcaccattcagca

SEQ ID NO: 2 - DNA, 50 bp, Artificial Sequence
  Reverse primer for alpha A-crystallin
ggggaccactttgtacaagaaagctgggtttcaggacgagggtgcagagc

SEQ ID NO: 3 - protein, 173 aa, Artificial Sequence
  AAH85172 for alpha A-crystallin
MDVTIQHPWFKRALGPFYPSRLFDQFFGEGLFEYDLLPFLSSTISPYYRQSLFRTVLDSGISEVRSDRDKFVIFLDVKHFSPEDLTVKVLEDFVEIHGKHNERQDDHGYISREFHRRYRLPSNVDQSALSCSLSADGMLTFSGPKVQSGLDAGHSERAIPVSREEKPSSAPSS

SEQ ID NO: 4 - DNA, 522 bp, Artificial Sequence
  BC085172 for alpha A-crystallin
atggacgtcaccattcagcatccttggttcaagcgtgccctggggcccttctaccccagccgactgttcgaccagttcttcggcgagggcctttttgagtacgacctgctgcccttcctgtcttccaccatcagcccctactaccgccagtccctcttccgcactgtgctggactcgggcatctctgaggtccgatctgaccgggacaagtttgtcatcttcttggacgtgaagcacttctctcctgaggacctcaccgtgaaggtactggaggattttgtggagattcacggcaaacacaacgagaggcaggatgaccatggctacatttcccgtgaatttcaccgtcgctaccgtctgccttccaatgtggaccagtccgccctctcctgctccctgtctgcggatggcatgctgaccttctctggccccaaggtccagtccggtttggatgctggccacagcgagagggccattcctgtgtcacgggaggagaaacccagctctgcaccctcgtcctga